(19) United States
(12) Patent Application Publication
(10) Pub. No.: US 2026/0090730 A1
(43) Pub. Date: Apr. 2, 2026

(54) NOTIFICATION APPARATUS

(71) Applicant: ISUZU MOTORS LIMITED, Yokohama-shi (JP)

(72) Inventor: Mayumi IWAO, Fujisawa-shi (JP)

(21) Appl. No.: 19/299,919

(22) Filed: Aug. 14, 2025

(30) Foreign Application Priority Data
Sep. 27, 2024 (JP) 2024-168490

(51) Int. Cl. A61B 5/0245

(52) U.S. Cl. CPC A61B 5/02455; A61B 2503/20; A61B 2503/22

(57) ABSTRACT

A notification apparatus includes: a storage unit that stores first information in which a first range of activity levels in a case where a worker is in one state is associated with a second range of heart rates in a case where the worker is in the one state; an acquisition unit that acquires the activity level per unit time of the worker during work and heart rate per unit time of the worker during the work; and a notification control unit that notifies second information related to the one state when the acquired activity level is within the first range and the acquired heart rate is within the second range.

### CROSS-REFERENCE TO RELATED APPLICATIONS

The present application claims priority to Japanese Patent Application No. 2024-168490, filed on September 27, 2024, contents of which are incorporated herein by reference in their entirety.

### BACKGROUND OF THE INVENTION

The present disclosure relates to a notification apparatus that provides, as notification, a state of a worker engaged in work. Techniques for providing notification of the state of a worker are known. For example, Japanese Unexamined Patent Application Publication No 2022-102177 discloses a technique for providing notification of the degree of fatigue based on the maximum heart rate at when a worker has fallen asleep.

However, in the technique disclosed in Japanese Unexamined Patent Application Publication No 2022-102177, while the degree of fatigue during sleep can be grasped, it has not been possible to grasp what state the worker is in during work. In order to appropriately manage the worker’s tasks, a technique for grasping the state of the worker during work has been required.

### BRIEF SUMMARY OF THE INVENTION

The present disclosure has been made in view of these points, and its object is to grasp a state of a worker during work.

An aspect of the present disclosure provides a notification apparatus including: a storage unit that stores first information, in which a first range of activity levels in a case where a worker during work is in one of a plurality of states and a second range of heart rates in a case where the worker is in the one state are associated with each other; an acquisition unit that acquires activity level per unit time of the worker during the work and heart rate per unit time of the worker during the work; and a notification control unit that notifies second information relating to the one state when the acquired activity level is within the first range and the acquired heart rate is within the second range.

### BRIEF DESCRIPTION OF THE DRAWINGS

FIG. 1 is a diagram for explaining an overview of a notification system.

FIG. 2 is a diagram in which the acquired activity level and heart rate are plotted.

FIG. 3 is a diagram illustrating a configuration of a notification apparatus.

FIG. 4 is a diagram for explaining a region represented by a range of activity levels and a range of heart rates.

FIG. 5 is a schematic diagram of a display screen displayed on an administrator terminal.

FIG. 6 is a flowchart showing an example of a process of providing, as notification, second information.

### DETAILED DESCRIPTION OF THE INVENTION

Hereinafter, the present disclosure will be described through exemplary embodiments, but the following exemplary embodiments do not limit the invention according to the claims, and not all of the combinations of features described in the exemplary embodiments are necessarily essential to the solution means of the invention.

### First Embodiment

### An overview of a notification system S

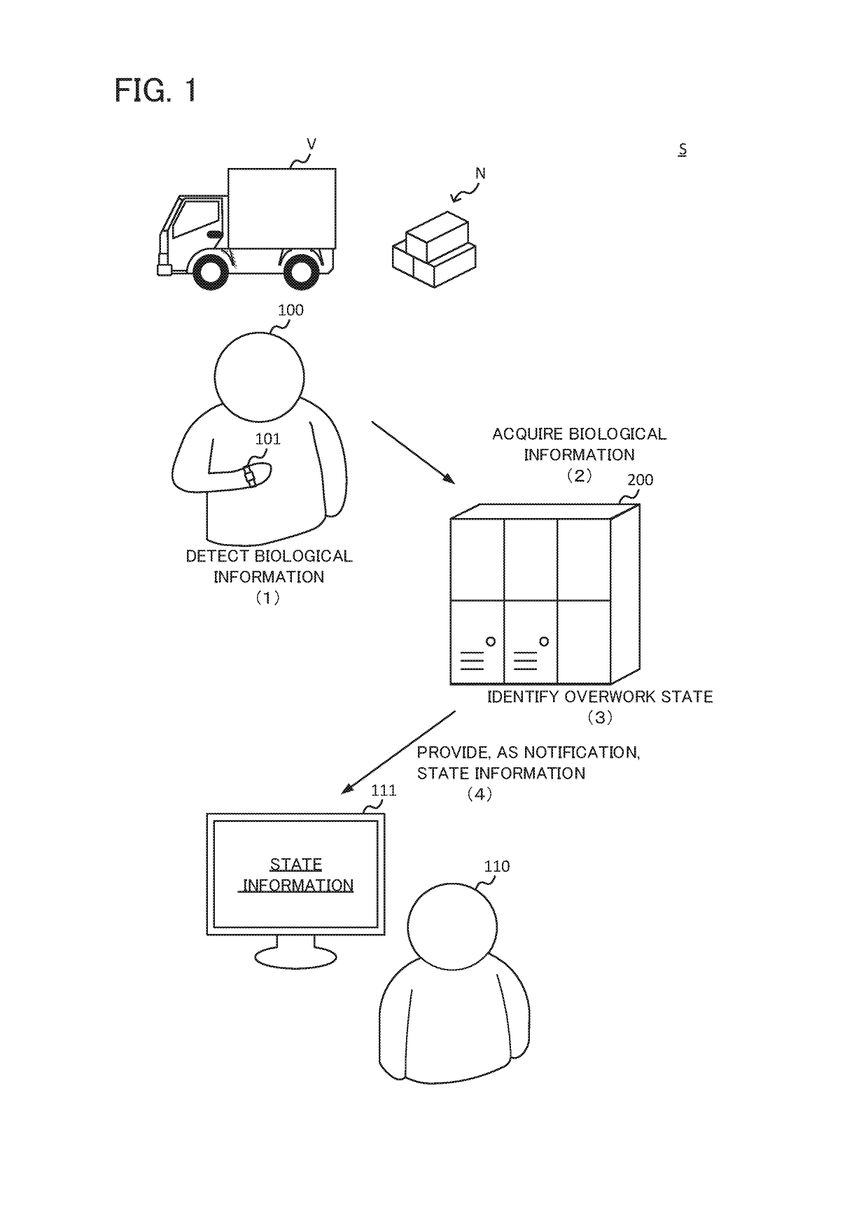

FIG. 1 is a diagram for explaining an overview of a notification system S. The notification system S is a system that notifies an administrator 110, who manages a worker 100, of a state of the worker 100. The notification system S includes a smartwatch 101, an administrator terminal 111, and a notification apparatus 200.

The worker 100 is a driver of a vehicle V that transports articles N. The vehicle V is, for example, a truck or a trailer. The worker 100 performs a cargo handling operation for loading and unloading the articles N to and from the vehicle V. The worker 100 wears a mobile device capable of detecting biological information of the worker 100. The worker 100 according to the present embodiment wears the smartwatch 101, as the mobile device, on his/her arm.

The smartwatch 101 detects biological information of the worker 100 ((1) in FIG. 1). The smartwatch 101 detects the activity level of the worker 100 during work as biological information. The activity level of the worker 100 is represented by a ratio of the energy consumption of the worker 100 in a given state to the energy consumption of the worker 100 at rest. The energy consumption of the worker 100 increases when the worker 100 is performing tasks with a high physical load. The motion of the worker 100 becomes greater when he/she is performing the tasks with a high physical load. When the motion of the worker 100 increases, acceleration applied to the smartwatch 101 increases.

Using this fact, the smartwatch 101 detects the energy consumption on the basis of acceleration. The smartwatch 101 includes an acceleration sensor capable of measuring acceleration in three directions, and identifies the energy consumption per unit time of the worker 100 during work on the basis of acceleration in each direction. The unit time is 10 minutes, but is not limited thereto. The smartwatch 101 identifies greater energy consumption as the magnitude of a vector obtained by synthesizing the three accelerations increases. The smartwatch 101 identifies, as the activity level, a ratio of the identified energy consumption to the energy consumption of the worker 100 at rest. It should be noted that the energy consumption of the worker 100 at rest is assumed to be stored in the smartwatch 101 in advance.

The smartwatch 101 detects the heart rate of the worker 100 during work as the biological information. The smartwatch 101 includes an optical heart rate sensor, and detects a heart rate per unit time of the worker 100 during work on the basis of a change in reflected light when the skin of the worker 100 is irradiated with green light. The smartwatch 101 may detect the heart rate by another method.

The smartwatch 101 detects the activity level and heart rate at predetermined detection intervals. The detection interval is, for example, one minute. The smartwatch 101 detects the activity level and heart rate at the same timing every minute. The smartwatch 101 detects the activity level and heart rate per unit time longer than the detection interval. The unit time is, for example, 10 minutes. The smartwatch 101 detects the activity level and heart rate per unit time on the basis of a statistical value of ten activity levels detected during the 10 minutes and a statistical value of ten heart rates. The statistical value may be at least one of a mean value, a median, and a mode. The smartwatch 101 according to the present embodiment sets a mean value of the ten activity levels and a mean value of the ten heart rates detected during 10 minutes as the activity level and heart rate per unit time

The smartwatch 101 includes a wireless communication module, and is connected to the notification apparatus 200 in a manner enabling communication. The smartwatch 101 transmits biological information indicating the detected activity level and heart rate per unit time to the notification apparatus 200.

The notification apparatus 200 is a server managed by the administrator 110. The notification apparatus 200 stores biological information of the worker 100 during work, tasks scheduled to be performed by the worker 100 during the work period, contents of tasks actually performed by the worker 100 during the work period, and the like.

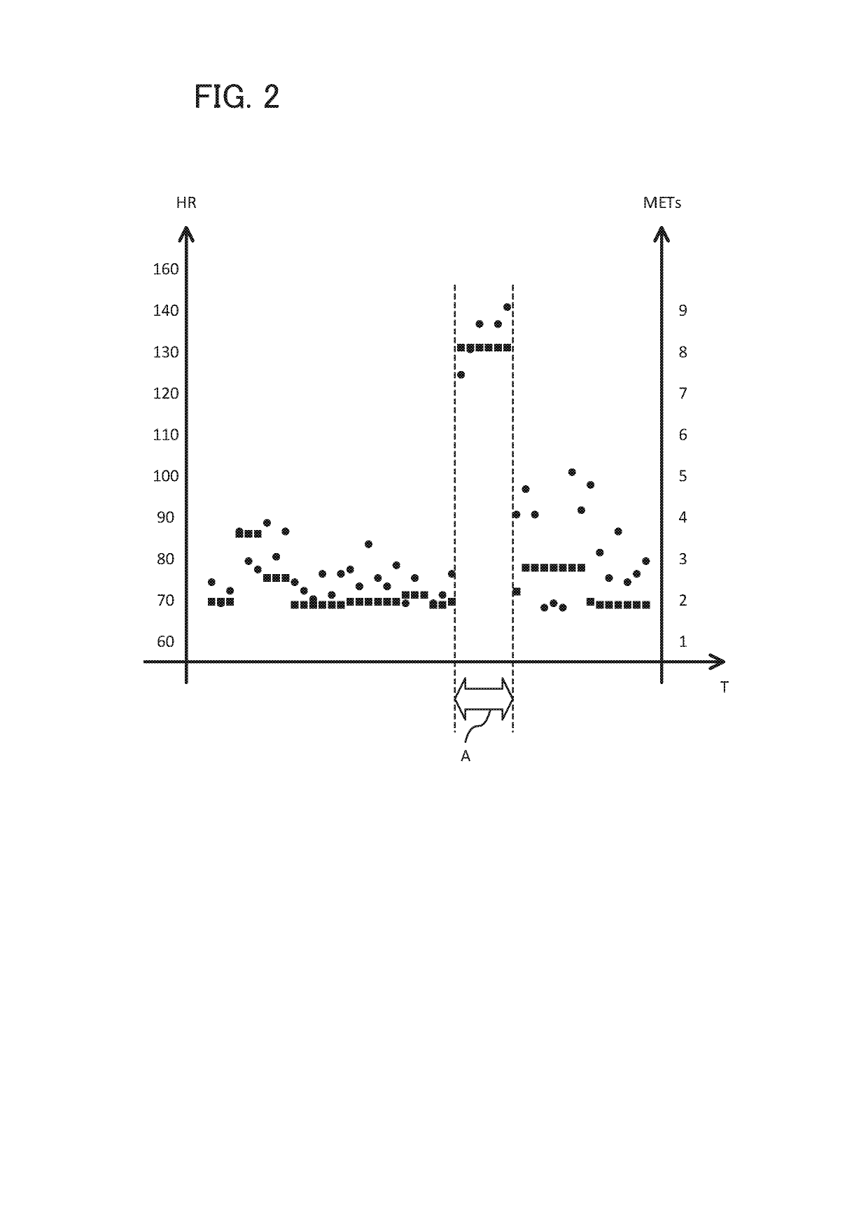

The notification apparatus 200 acquires biological information indicating the activity level and heart rate from the smartwatch 101 ((2) in FIG. 1). FIG. 2 is a diagram in which the acquired activity level and heart rate are plotted. In FIG. 2, the horizontal axis indicates time T, the left vertical axis indicates the heart rate (HR), and the right vertical axis indicates the activity level (METs). In FIG. 2, the mean value of the heart rate of the worker 100 during tasks over each 10-minute interval was plotted with circles, and the mean value of the activity level over each 10-minute interval was plotted with squares.

As described above, it can be said that the higher the activity level, the more the worker 100 is performing tasks with a high physical load. The activity level in period A is “8”, which is higher than the activity level in periods other than the period A, and it is considered that the worker 100 has continuously performed tasks with a high physical load during the period A. The heart rate in the period A, in which the worker 100 has performed tasks with a high physical load, is higher than the heart rate at time T outside the period A. It is considered that the heart rate in the period A became high due to an excessive load applied to the body of the worker 100 as a result of the worker 100 performing tasks with a high physical load. In the period A, during which the activity level and heart rate of the worker 100 are high, it is considered that the state of the worker 100 was an overwork state. The overwork state is a state in which an excessive load is applied to the worker 100 by performing tasks with a high physical load.

As described above, it is possible to identify that the worker 100 has been in the overwork state during the work period on the basis of the activity level and heart rate of the worker 100. The notification apparatus 200 identifies an overwork state of the worker 100 during work on the basis of the activity level and heart rate acquired from the smartwatch 101 ((3) in FIG. 1). For example, when activity levels and heart rates of the worker 100 are within predetermined ranges, the notification apparatus 200 identifies that the worker 100 is in the overwork state. Specifically, when the activity level is within a range of activity levels corresponding to the overwork state and the heart rate is within a range of heart rates corresponding to the overwork state, notification apparatus 200 identifies that the worker 100 is in the overwork state.

The notification apparatus 200 provides, as notification, state information regarding the state of the worker 100 ((4) in FIG. 1). For example, the notification apparatus 200 provides, as notification, the state information (second information) related to the identified overwork state. The notification apparatus 200 provides, as notification, the second information by causing the administrator terminal 111 to display an image or text related to the identified overwork state as the second information. The administrator terminal 111 is a terminal of the administrator 110. By reviewing the image or text related to the overwork state, which is the second information displayed on the administrator terminal 111, the administrator 110 becomes able to grasp that the worker 100 was in the overwork state during the work period. As a result, since it becomes easier to adjust the workload of the worker 100 and perform health management for the worker 100, the administrator 110 can appropriately manage the work of the worker 100. Hereinafter, a specific configuration of the notification apparatus 200 will be described.

### Configuration of the notification apparatus 200

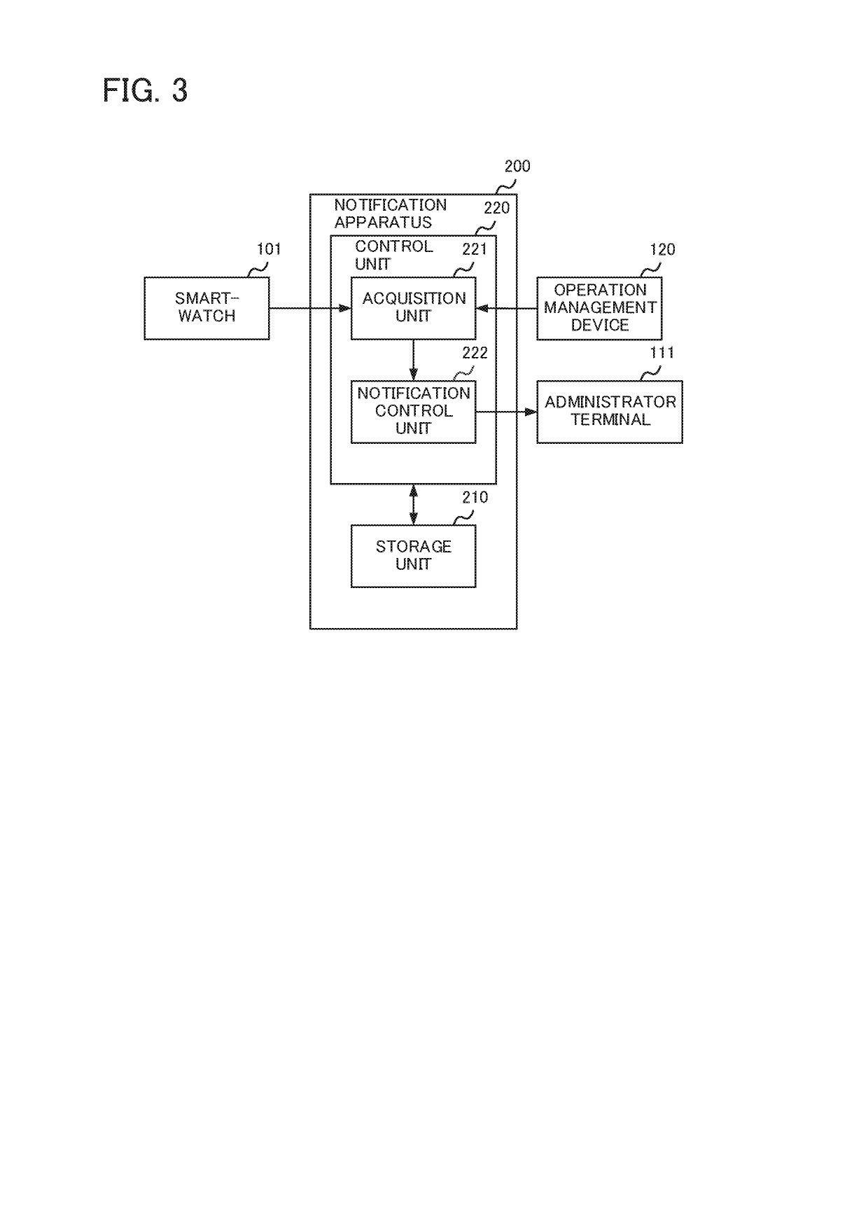

FIG. 3 is a diagram illustrating a configuration of the notification apparatus 200. The notification apparatus 200 includes a storage unit 210 and a control unit 220. The storage unit 210 is a storage medium such as a read only memory (ROM), a random access memory (RAM), a hard disk, and the like.

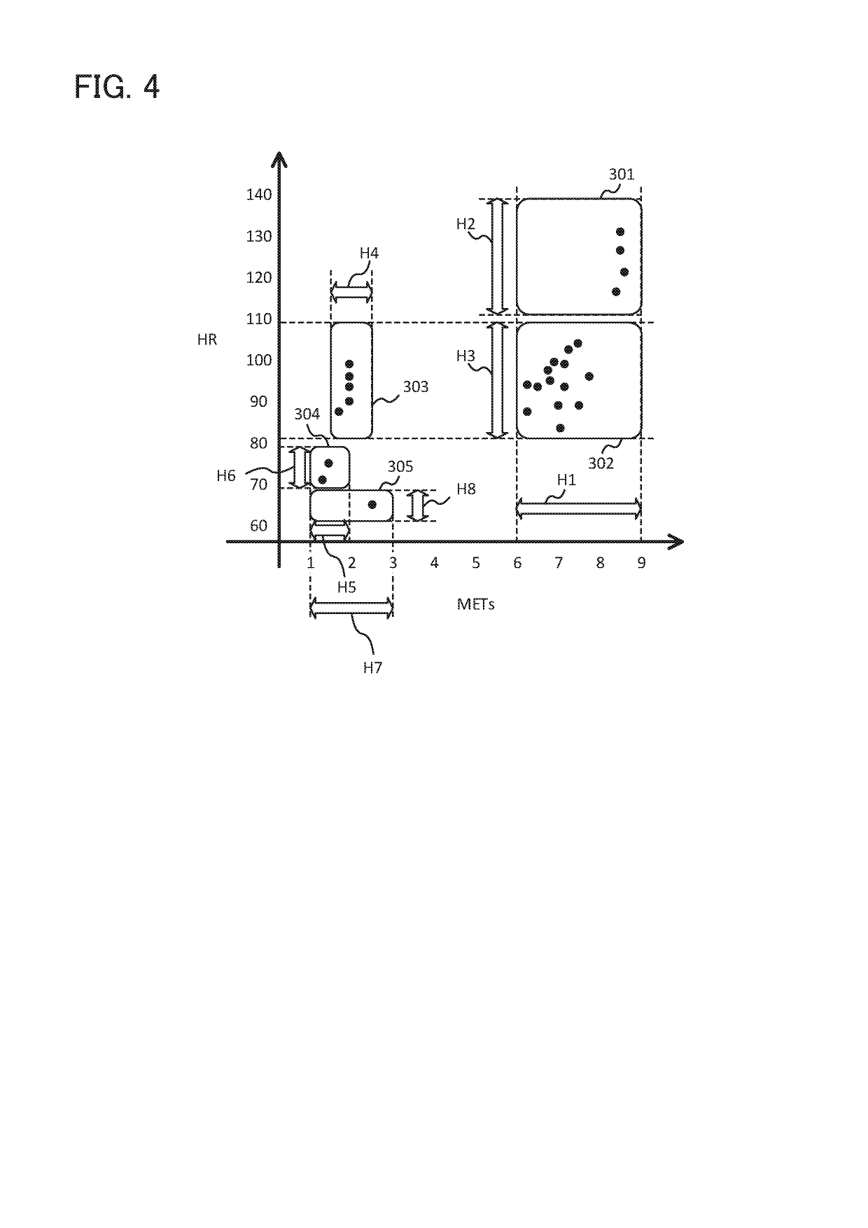

The storage unit 210 stores first information for identifying the state of the worker 100. For example, the storage unit 210 stores the first information in which a range of activity levels and a range of heart rates, both corresponding to one of a plurality of states of the worker 100, are associated with each other. Specifically, the storage unit 210 stores the first information indicating a region represented by a range of activity levels and a range of heart rates, both corresponding to each of the plurality of states of the worker 100 during work. FIG. 4 is a diagram for explaining the first information. In FIG. 4, the horizontal axis represents the activity level METs, and the vertical axis represents the heart rate HR. As illustrated in FIG. 4, the first information includes a plurality of regions corresponding to the plurality of states.

A first region 301 is a region corresponding to a first state. The first state is a state in which the worker 100 is in a high-activity state, wherein the worker 100 is performing tasks involving a high activity level, and is also in a high-load state where the load on the worker 100 is high. In other words, the first state is a state in which an excessive load is being applied to the worker 100 who is performing tasks with a high physical load. The first region 301 is a region in which the activity level is within a first range H1 and the heart rate is within a second range H2.

The first range H1 is a range of activity levels when the worker 100 is in the high-activity state. The first range H1 is a range in which the activity level is higher than or equal to a first predetermined value and lower than or equal to a predetermined upper limit value. The first predetermined value is a value for determining that the worker 100 is performing tasks with a high physical load and involving a high activity level. Tasks with a high physical load include, for example, a cargo handling operation for loading and unloading heavy items. A specific value of the first predetermined value is 6, but is not limited thereto. The predetermined upper limit value may be defined based on the activity level when the worker 100 performs the cargo handling operation, and is, for example, 9, but is not limited thereto.

The second range H2 of the heart rate is a range of heart rates in the high-load state in which a high physical load is being applied to the worker 100. The second range H2 of the heart rate is a range equal to or greater than a second predetermined value. The second predetermined value is a value for determining that the worker 100 is in the high-load state. The second predetermined value is defined based on the maximum heart rate of the worker 100, for example, 60% of the maximum heart rate, which would be 110. However, the value is not limited thereto, and may instead be defined based on the maximum heart rate of the worker 100 managed by the administrator 110.

A second region 302 is a region corresponding to a second state. The second state is a state in which the worker 100 is in the high-activity state and also in a medium-load state, where the load on the worker 100 is lower than in the high-load state. The second state is a state in which a moderate physical load is being applied to the worker 100 who is in the high-activity state. The range of activity levels of the second region 302 is represented by the first range H1. The reason why the range of activity levels of the second region 302 is represented by the first range H1 is that the activity level in the second state becomes approximately the same as the activity level in the first state. The heart rate range of the second region 302 is represented by a third range H3. Since the heart rate when the worker 100 is in the medium-load state is lower than the heart rate when the worker 100 is in the high-load state, the upper limit value of the third range H3 is lower than the lower limit value of the second range H2 in the high-load state. The third range H3 according to the present embodiment is a range in which the heart rate is greater than or equal to 85 and less than 110.

The third region 303 is a region corresponding to a third state. The third state is a state in which the worker 100 is in a low-activity state, where the worker 100 is performing tasks involving an activity level lower than that in the high-activity state, and is also in the medium-load state. The third state is a state in which the worker 100 is in poor physical condition due to fever, poor sleep, chronic fatigue, or the like.

The range of the activity level of the third region 303 is represented by a fourth range H4. The activity level when the worker 100 is in the low-activity state is lower than the activity level when the worker 100 is in the high-activity state. Therefore, the upper limit value of the fourth range H4 is lower than the lower limit value of the first range H1 of the activity level in the high-activity state. The fourth range H4 according to the present embodiment is a range in which the activity level is equal to or greater than 1.5 and equal to or less than 2.5. The heart rate range of the third region 303 is represented by a third range H3 of the heart rate in the medium-load state. The reason why the heart rate range of the third region 303 is represented by the third range H3 is that the heart rate increases to the same level as that in the medium-load state even when the worker 100 is in the low-activity state in a case where the worker 100 is in poor physical condition due to fever, poor sleep, chronic fatigue, or the like.

The fourth region 304 is a region corresponding to a fourth state. The fourth state is an inactive state in which the worker 100 is not moving the body, and also in a low-load state, where the load on the worker 100 is low. That is, the fourth state is a state in which the heart rate is high despite inactivity caused by fever, poor sleep, chronic fatigue, or the like.

The range of the activity level of the fourth region 304 is represented by a fifth range H5. The activity level when the worker 100 is in the inactive state is lower than the activity level when the worker is in the low-activity state. Therefore, the upper limit value of the fifth range H5 is lower than the upper limit value of the fourth range H4 corresponding to the low-activity state, and the lower limit value of the fifth range H5 is lower than the lower limit value (1.5) of the fourth range H4. The fifth range H5 according to the present embodiment is a range in which the activity level is equal to or greater than 1 and less than 2.

The heart rate range of the fourth region 304 is represented by a sixth range H6. The heart rate in the inactive state when the physical condition of the worker 100 is poor is lower than the heart rate in the high-load state. Therefore, the upper limit value of the sixth range H6 is lower than the lower limit value (85) of the third range H3 of the heart rate in the high-load state. Furthermore, since the heart rate in the inactive state when the physical condition of the worker 100 is poor is higher than the heart rate in the inactive state when the physical condition is good, the lower limit value of the sixth range H6 is higher than the upper limit value (68) of the heart rate range in the inactive state when the physical condition is good. The sixth range H6 according to the present embodiment is a range in which the heart rate is greater than 68 and less than 80.

The fifth region 305 is a region corresponding to the fifth state. The fifth state is a state in which the worker 100 is in the low-activity state and also in an extremely low-load state, where the load on the worker 100 is lower than in the low-load state. In other words, the fifth state is a state in which an alertness level of the worker 100 during tasks is low, and is, for example, a state close to dozing or a state indicating a tendency toward decreased alertness, such as beginning to doze off.

The range of the activity level of the fifth region 305 is represented by a seventh range H7. Low-activity tasks are tasks with a lower physical load than tasks with a high physical load, and for example, the low-activity tasks are, for example, driving or inspecting the vehicle V, a cargo handling operation for loading or unloading light items, desk work, or the like, but are not limited thereto.

Since the activity level when the worker 100 is performing low-activity tasks is lower than the activity level during performing tasks with a high physical load, the upper limit value of the seventh range H7 is lower than the lower limit value (6) of the first range H1 of the activity level in the high-activity state. In addition, the upper limit value of the seventh range H7 is higher than the upper limit value (2) of the fourth range H4 during performing tasks with a low physical load. This is because, when the worker 100 is performing low-activity tasks such as driving the vehicle V or doing desk work, the worker 100 may move their arm significantly, and acceleration associated with the smartwatch 101 becomes greater than that in the case of performing tasks with a low physical load. Since the activity level in the low-activity state may be approximately the same as the activity level at rest, the lower limit value of the seventh range H7 is the activity level (1) at rest. The seventh range H7 according to the present embodiment is a range in which the activity level is equal to or greater than 1 and equal to or less than 3.

The heart rate range of the fifth region 305 is represented by an eighth range H8. The heart rate when the worker 100 is in the extremely low-load state is lower than the heart rate when the worker 100 is active or the heart rate in the inactive state of the worker 100 in which the physical condition is poor. Therefore, the upper limit value of the eighth range H8 is lower than the lower limit value (68) of the sixth range H6 of the heart rate in the low-load state. The lower limit value of the eighth range H8 is the lowest value among the values measured as the heart rate of the worker 100 in the inactive state. The eighth range H8 according to the present embodiment is a range in which the heart rate is equal to or greater than 60 and less than 68.

The storage unit 210 stores programs executed by the control unit 220. The control unit 220 is a calculation resource including a processor such as a central processing unit (CPU). By executing the programs stored in the storage unit 210, the control unit 220 functions as an acquisition unit 221 and a notification control unit 222.

The acquisition unit 221 acquires the activity level and heart rate of the worker 100 during work. The acquisition unit 221 acquires the activity level and heart rate detected by the smartwatch 101 at predetermined intervals. The predetermined interval is, for example, 10 minutes, which is the same as the unit time, but is not limited thereto.

The notification control unit 222 identifies the state of the worker 100 on the basis of the activity level and heart rate. When the acquired activity level and heart rate fall within any of the plurality of regions indicated in the first information, the notification control unit 222 identifies that the state of the worker 100 corresponds to one region including the activity level and heart rate. The one region is defined by a range of activity levels and a range of heart rates, and is a region to which a given state is associated. For example, when the acquired activity level is within the range of activity levels associated with one region, and the acquired heart rate is within the range of heart rates associated with that same region, the notification control unit 222 identifies that the state corresponds to the given state associated with that region. As a specific example, when the acquired activity level is within the first range H1 and the acquired heart rate is within the second range H2, the notification control unit 222 identifies that the worker 100 is in the first state corresponding to the first region 301.

The notification control unit 222 identifies the state of the worker 100 on the basis of the duration during which the acquired activity level and heart rate fall within one of the plurality of regions. For example, when the duration during which the acquired activity level and heart rate fall within one of the plurality of regions reaches or exceeds a determination duration corresponding to that region, the notification control unit 222 identifies that the state of the worker 100 corresponds to the one region including the activity level and heart rate. The determination duration is the time for determining that the worker 100 is in the given state, and is set for each of the plurality of regions. The higher the range of the activity levels and the range of the heart rates, the shorter the determination duration. Hereinafter, a process in which the notification control unit 222 identifies the state on the basis of the determination duration will be described using a specific example.

When a duration during which the activity level is within the first range H1 and the heart rate has been within the second range H2 reaches or exceeds a first determination duration corresponding to the first region 301, the notification control unit 222 identifies that the worker 100, who has remained in the first state, has entered the overwork state. The first determination duration is the time for determining that the first state is continuing. The first determination duration is, for example, 40 minutes, but is not limited thereto.

When a duration during which the activity level and heart rate have been within the second region 302 reaches or exceeds a second determination duration corresponding to the second region 302, the notification control unit 222 identifies that the worker 100 is in the second state corresponding to the second region 302. The second determination duration is the time for determining that the second state is continuing. The second determination duration is longer than the first determination duration and is, for example, 60 minutes, but is not limited thereto.

In this manner, the notification control unit 222 can appropriately identify the second state, which tends to continue for a longer time than the overwork state.

When a duration during which the activity level and heart rate have been within the third region 303 reaches or exceeds a third determination duration corresponding to the third region 303, the notification control unit 222 identifies that the worker 100 is in the third state corresponding to the third region 303. Similarly, when a duration during which the activity level and heart rate have been within the fourth region 304 reaches or exceeds a fourth determination duration corresponding to the fourth region 304, the notification control unit 222 identifies that the worker 100 is in the fourth state corresponding to the fourth region 304. The third determination duration and the fourth determination duration are shorter than the first determination duration. Specifically, when it is identified that the worker 100 is in the third state or the fourth state, the notification control unit 222 identifies that the worker 100 is in poor physical condition. The third determination duration and the fourth determination duration may be the same or different. The third determination duration and the fourth determination duration may be appropriately determined by experiments or the like, and are, for example, 30 minutes, but are not limited thereto. The third determination duration and the fourth determination duration may be changeable.

When a duration during which the activity level and heart rate have been within the fifth region 305 reaches or exceeds the fifth determination duration corresponding to the fifth region 305, the notification control unit 222 identifies that the worker 100 is in the fifth state corresponding to the fifth region 305. The fifth determination duration is shorter than the first determination duration and is, for example, 5 minutes. When the notification control unit 222 identifies that the worker 100 is in the fifth state, it identifies that the worker 100 is in a low alertness state. In this manner, since the notification control unit 222 can appropriately identify the fifth state, which tends to continue for a shorter time than the other states, it becomes easier to identify the low alertness state, such as a state close to dozing or beginning to doze off during driving.

The notification control unit 222 identifies the state of the worker 100 on the basis of the activity level and heart rate acquired during a predetermined period within a work period of the worker 100. The work period refers to the period from a work start time, at which the worker 100 begins work, to a work end time, at which the worker 100 finishes work. The notification control unit 222 divides the work period into a plurality of periods. For example, the notification control unit 222 divides the work period into the plurality of periods by dividing the work period from the work start time at predetermined intervals. Specifically, the notification control unit 222 divides the work period (10 hours) into ten periods by dividing the work period from the work start time (e.g., 7:00AM) at predetermined intervals (e.g., every hour).

The notification control unit 222 identifies the state of the worker 100 on the basis of an integrated value representing the total duration during which the activity level and heart rate acquired in one of the ten periods of the work period fall within one of the plurality of regions. When the integrated value, which is the product of the number of times the activity level and heart rate acquired in one of the ten periods fall within one region and the unit time, is equal to or greater than the determination duration corresponding to that region, the notification control unit 222 identifies that the worker 100 has been in the state corresponding to the one region. In this way, the notification control unit 222 can identify the state of the worker 100 in each of the plurality of periods into which the work period is divided.

The notification control unit 222 identifies tasks performed by the worker 100 in a given state. In this case, the acquisition unit 221 acquires the biological information indicating the activity level and heart rate, in association with the time at which the activity level and heart rate are detected. In addition, the acquisition unit 221 acquires, from an operation management device 120, operation management information in which (i) tasks scheduled to be performed by the worker 100 and (ii) the time at which the tasks scheduled to be performed are associated with each other. The operation management device 120 is a server that stores the operation management information. The administrator 110 inputs the tasks scheduled to be performed by the worker 100 and the time at which the tasks scheduled to be performed to the operation management device 120 via the administrator terminal 111. The operation management device 120 stores the operation management information input from the administrator 110.

The notification control unit 222 references the operation management information acquired by the acquisition unit 221, and identifies tasks corresponding to the acquired activity level and the time at which the heart rate is detected. Specifically, the notification control unit 222 identifies tasks that have been performed at the time when the activity level and heart rate used for identifying the state of the worker 100 are detected. For example, the notification control unit 222 identifies that the task that has been performed at the time when the activity level and heart rate used to identify that the worker 100 is in the overwork state is the cargo handling operation.

The notification control unit 222 provides, as notification, state information (second information) related to the identified state. The state information is an image or text indicating that the worker 100 is in the identified state. When the worker 100 is identified as being in the first state (overwork state), the notification control unit 222 provides, as the state information, notification that is an image or text indicating that the worker 100 was in the overwork state. When at least one of the second state, the third state (poor physical condition), the fourth state (poor physical condition), and the fifth state (low alertness state) is identified in addition to the overwork state, the notification control unit 222 provides, as the state information, an image or text indicating that the worker 100 was in the identified state. Additionally, in a case where a plurality of states are identified, the notification control unit 222 provides, as notification, state information that includes an image or text indicating that the worker 100 was in each of the plurality of identified states. The notification control unit 222 provides the administrator 110 with the state information by causing the administrator terminal 111 to display, at the end of the worker 100’s work, an image or text indicating that the worker 100 was in the identified state, as the state information. The administrator 110 can grasp the state of the worker 100 during work by checking the image or text which is the state information displayed on the administrator terminal 111.

The notification control unit 222 may provide, as notification, the state information related to a given state by associating the task performed in the given state with the state information related to that given state. For example, the notification apparatus 200 causes the administrator terminal 111 to display an image or text indicating that the task that has been performed in the overwork state was the cargo handling operation. As a result, the administrator 110 can grasp what kind of task was being performed when the worker 100 enters the overwork state.

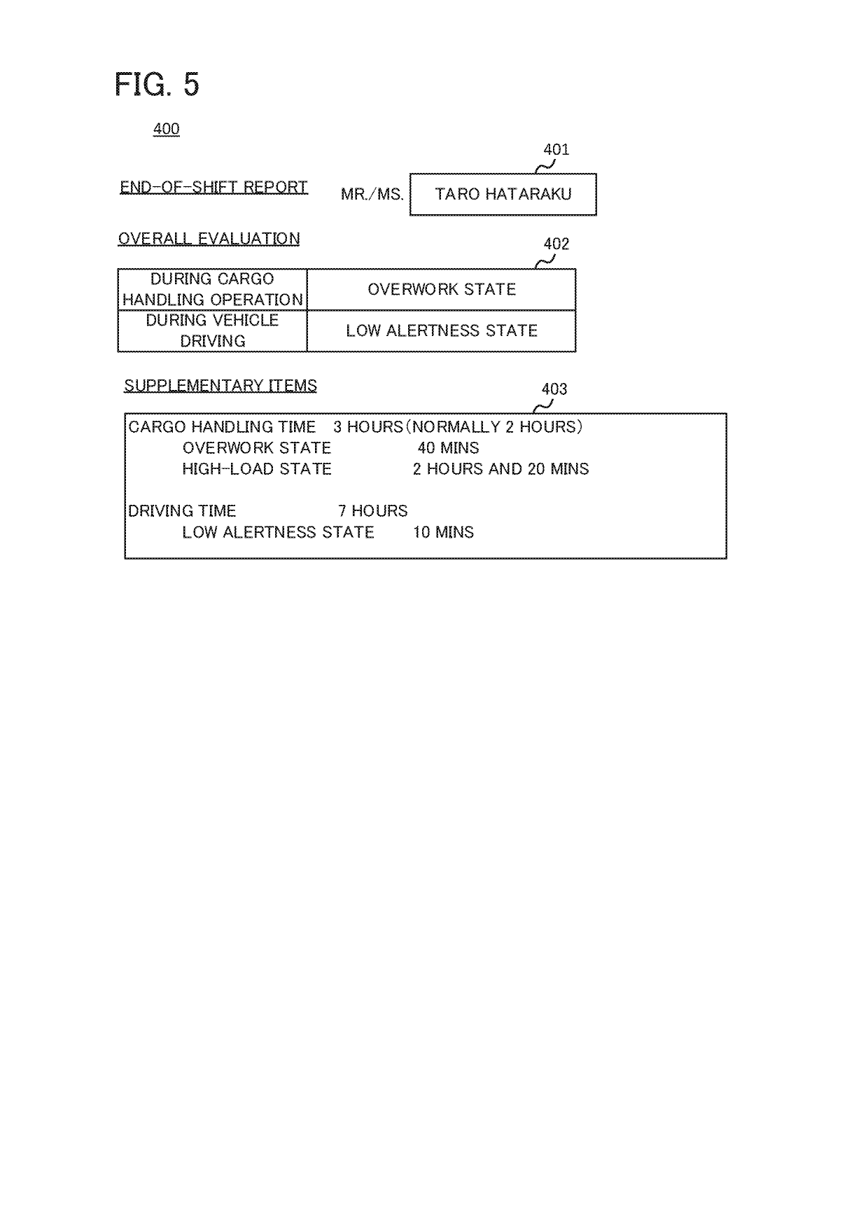

FIG. 5 is a schematic diagram of a display screen 400 displayed on the administrator terminal 111. The display screen 400 is a display screen showing state information related to the identified state. On the display screen 400, an image or text indicating that the worker 100 was in the identified state is displayed as the state information. The name of the worker 100 is displayed in a name field 401. In this way, the administrator 110, who manages a plurality of workers 100, can grasp which worker 100’s information is being confirmed.

In an overall evaluation field 402, the state during the cargo handling operation and a state during vehicle driving are displayed. In the overall evaluation field 402 of FIG. 5, it is also displayed that the overwork state continued during the cargo handling operation and that the low alertness state continued during vehicle driving. The administrator 110 can grasp the state of the worker 100 during work by checking the state of the worker 100 displayed in the overall evaluation field 402.

In the supplementary item field 403, the cargo handling time during which the cargo handling operation was performed, the driving time during which the vehicle V was driven, and the time during which the worker 100 was in a given state are displayed. In a supplementary item field 403 of FIG. 5, it is displayed that the cargo handling time is 3 hours and that the normal cargo handling time is 2 hours. In addition, the supplementary item field 403 of FIG. 5 displays the duration of the overwork state and the duration of the high-load state within the cargo handling time. Specifically, the supplementary item field 403 of FIG. 5 displays that, out of the 3-hour cargo handling time, 40 minutes correspond to the overwork state and 2 hours and 20 minutes correspond to the high-load state. In this manner, the administrator 110 can grasp the details of the state of the worker 100 who has performed the cargo handling operation.

In addition, the supplementary item field 403 displays the driving time during which the worker 100 drove the vehicle V. The supplementary item field 403 of FIG. 5 displays that the driving time was 7 hours. In a case where the worker 100 enters the low alertness state while driving, the supplementary item field 403 displays the duration of the low alertness state within the driving time. As described above, since a given state of the worker 100 during work and the duration during which the worker 100 was in the given state are displayed on the administrator terminal 111, the administrator 110 can grasp how long the worker 100 was in a particular state and what that state was during the work period.

### Process for providing, as notification, state information

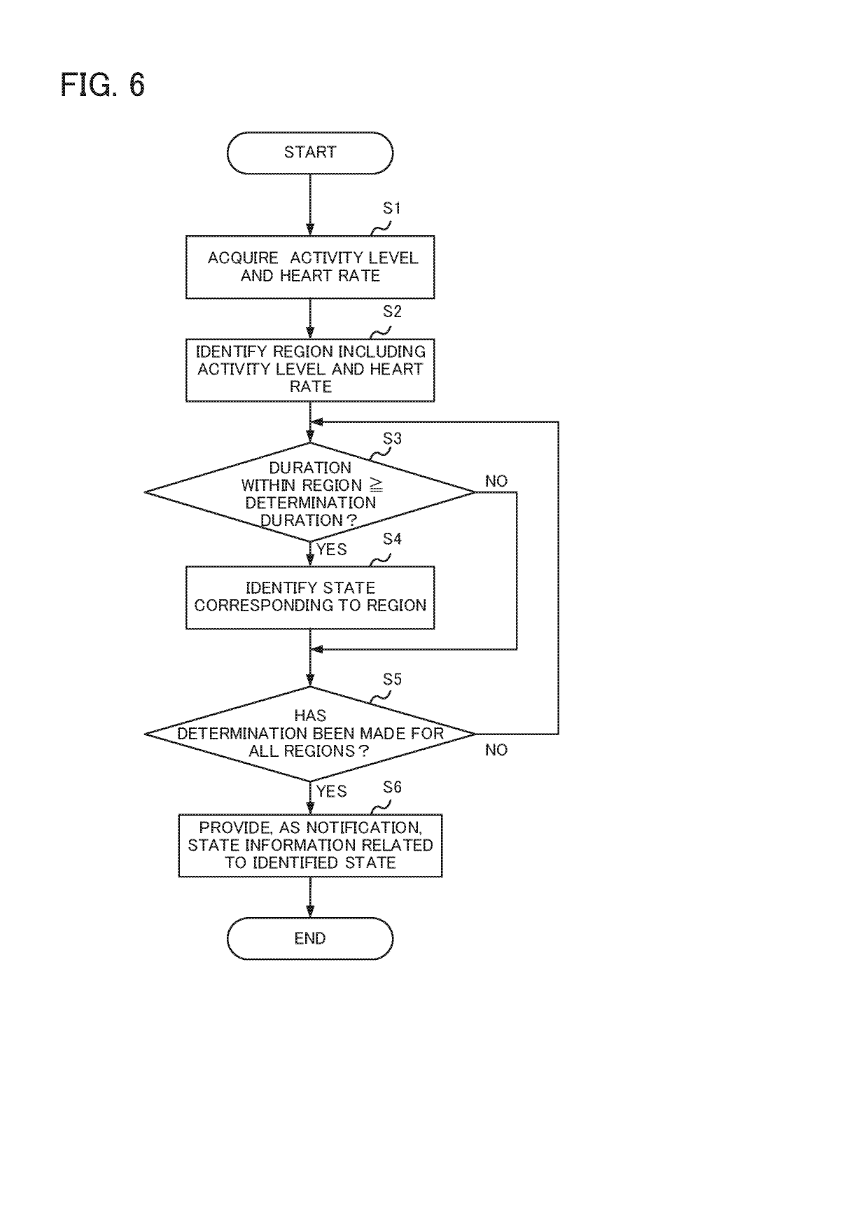

FIG. 6 is a flowchart illustrating an example of a process for providing, as notification, state information. The process for providing the state information as notification is executed at the end of the worker 100’s work.

When the work of the worker 100 is completed, the acquisition unit 221 acquires the activity level and heart rate per unit time during work (step S1). Specifically, the acquisition unit 221 acquires, during the worker period of the worker 100, the activity level and heart rate per unit time at predetermined intervals, and stores the acquired activity level and heart rate in the storage unit 210. When the work of the worker 100 is completed, the acquisition unit 221 acquires the activity level and heart rate stored in the storage unit 210.

The notification control unit 222 identifies a region including the acquired activity level and heart rate (step S2). Specifically, the notification control unit 222 identifies which one of the plurality of regions illustrated in FIG. 4 includes the acquired activity level and heart rate. For example, when the acquired activity level is within the first range H1 and the acquired heart rate is within the second range H2, the notification control unit 222 identifies that the acquired activity level and heart rate fall within the first region 301.

The notification control unit 222 determines whether the duration within the identified region, including the acquired activity level and heart rate, reaches or exceeds the determination duration (step S3). Specifically, the notification control unit 222 determines whether the duration, represented by the product of the unit time and the number of times that the activity level and heart rate acquired in a predetermined period (one hour) during the worker 100’s work fall within the identified region, reaches or exceeds the determination duration corresponding to that identified region. In one example, the notification control unit 222 determines whether the product (40 minutes) of (i) the number of times (four times) that the activity level and heart rate acquired in one period among a plurality of periods into which the work period is divided fall within the first region 301 and (ii) the unit time (10 minutes) reaches or exceeds the first determination duration (40 minutes) corresponding to the first region 301.

If the duration within one of the plurality of regions reaches or exceeds the determination duration (Yes in step S3), the notification control unit 222 identifies a state corresponding to the region including the activity level and heart rate (step S4). For example, the notification control unit 222 identifies, as the state of the worker 100, the overwork state corresponding to the first region 301 including the activity level and heart rate. If the duration within one of the plurality of regions is less than the determination duration (No in step S3), the notification control unit 222 proceeds to step S5.

The notification control unit 222 determines whether the determination has been made for all the regions indicated in the first information (step S5). Specifically, the notification control unit 222 determines, for each of the regions indicated in the first information, whether a determination has been made about whether the duration during which the acquired activity level and heart rate fall within the region reaches or exceeds the determination duration. If there is a region for which the determination has not been made (No in step S5), the notification control unit 222 repeats steps S3 to S5 until the determination has been made for all the regions.

If the determination has been made for all the regions (Yes in step S5), the notification control unit 222 provides, as notification, the state information related to the identified state (step S6). For example, the notification control unit 222 provides the administrator 110 with the state information by causing the administrator terminal 111 to display an image or text related to the overwork state as the state information.

### Modification 1

In the embodiment, the notification control unit 222 identifies the state of the worker 100 in each of the plurality of periods into which the work period is divided. However, the embodiment is not limited to this. The notification control unit 222 may identify what state the worker 100 came to be in over the entire work period. In this case, when the product of (i) the number of times the activity level and heart rate acquired during the work period fall within one region and (ii) the unit time is equal to or greater than the determination duration corresponding to that region, the notification control unit 222 identifies that the worker 100 has been, during work, in the state corresponding to the one region. Accordingly, the notification control unit 222 can identify that the worker 100 is in the overwork state in the entire work period even if the worker 100 is not in the overwork state in one period, for example.

### Modification 2

The notification control unit 222 may set each determination duration. For example, the notification control unit 222 sets the determination duration corresponding to a given region on the basis of a statistical value of the duration during which the activity level and heart rate in each of a plurality of past periods fell within the given region. The statistical value may be any one of a mean value, a median, or a mode. Specifically, when the worker 100 starts work, the notification control unit 222 sets the determination duration on the basis of a mean value of durations in which the activity level and heart rate in each of a plurality of periods, acquired between the day before the work start date and a predetermined number of days prior to the day before, fell within a given region. As a specific example, when the latest activity level and heart rate are acquired on September 18, the notification control unit 222 sets, as the determination duration, a duration (40 minutes) obtained by adding a predetermined value (e.g., 10 minutes) to the average (e.g., 30 minutes) of the durations during which the activity level and heart rate in a given period fell within a given region, acquired over seven days from September 11 to September 17.

### Modification 3

The worker 100 is not limited to the driver. For example, the worker 100 may be a picker who picks up the articles N in a warehouse, a delivery person who delivers items on foot, or a worker who performs nursing care or cleaning.

### Effects of the notification apparatus 200

As described above, when both the activity level per unit time of the worker 100 during work is within the first range H1 of the activity level in the case where the worker 100 during work is in a given state and the heart rate per unit time of the worker 100 is within the second range H2 of the heart rate in the case where the worker is in the given state, the notification apparatus 200 notifies the state information related to that given state. By checking the state information provided as notification, the administrator 110 can grasp the state of the worker 100 during work.

The present disclosure is explained based on the exemplary embodiments. The technical scope of the present disclosure is not limited to the scope explained in the above embodiments and it is possible to make various changes and modifications within the scope of the disclosure. For example, all or part of the apparatus can be configured with any unit which is functionally or physically dispersed or integrated. Further, new exemplary embodiments generated by arbitrary combinations of them are included in the exemplary embodiments. Further, effects of the new exemplary embodiments brought by the combinations also have the effects of the original exemplary embodiments.

What is claimed is:

1. A notification apparatus comprising:
a storage unit that stores first information, in which a first range of activity levels in a case where a worker during work is in one of a plurality of states and a second range of heart rates in a case where the worker is in the one state are associated with each other;
an acquisition unit that acquires activity level per unit time of the worker during the work and heart rate per unit time of the worker during the work; and
a notification control unit that notifies second information relating to the one state when the acquired activity level is within the first range and the acquired heart rate is within the second range.

2. The notification apparatus according to claim 1, wherein the acquisition unit acquires activity level per unit time during the work and heart rate per unit time during the work at predetermined intervals, and
the notification control unit provides, as notification, the second information when a duration during which the acquired activity level is within the first range and the acquired heart rate is within the second range reaches or exceeds a determination duration for determining that the state is the one state.

3. The notification apparatus according to claim 2, wherein the notification control unit provides, as notification, the second information when a duration during which the activity level acquired within a predetermined period longer than the unit time during the work falls within the first range and the heart rate acquired within the predetermined period is within the second range reaches or exceeds the determination duration.

4. The notification apparatus according to claim 2, wherein the storage unit stores the first information in which the first range and the second range are associated with each other when the worker during the work is in each of the plurality of states, and
the notification control unit provides, as notification, the second information related to the one state when a duration during which the acquired activity level is within the first range corresponding to the one state of the plurality of states and the acquired heart rate is within the second range corresponding to the one state reaches or exceeds the determination duration.

5. The notification apparatus according to claim 2, wherein the first range is a range higher than or equal to a first predetermined value for determining that the worker is in a high-activity state in which the worker is performing tasks involving a high activity level,
the second range is a range higher than or equal to a second predetermined value for determining that the worker is in a high-load state in which a high load is being applied to the worker , and
the notification control unit provides, as notification, the second information related to a first state in which the worker is in the high-activity state and the high-load state when a duration during which the acquired activity level is within the first range and the acquired heart rate is within the second range reaches or exceeds the determination duration.

6. The notification apparatus according to claim 5, wherein the storage unit stores the first information associated with a third range of heart rates in a medium-load state, in which a load applied to the worker is lower than in the high-load state,
the third range is a range in which an upper limit value is lower than a lower limit value of the second range, and
the notification control unit provides, as notification, the second information related to a second state in which the worker is in the high-activity state and the medium-load state when the acquired activity level is within the first range and the acquired heart rate is within the third range.

7. The notification apparatus according to claim 6, wherein the storage unit stores the first information associated with a fourth range of activity levels in a case where the worker is in a low-activity state where the worker is performing tasks involving an activity level lower than in the high-activity state,
the fourth range is a range in which an upper limit value is lower than a lower limit value of the first range, and
the notification control unit provides, as notification, the second information related to a third state in which the worker is in the low-activity state and the medium-load state when the acquired activity level is within the fourth range and the acquired heart rate is within the third range.

8. The notification apparatus according to claim 7, wherein the storage unit stores the first information associated with (i) a fifth range of activity levels when the worker is in an inactive state in which the worker is not moving the body and (ii) a sixth range of heart rates, in which a load applied to the worker is lower than a load applied in the medium-load state,
the fifth range is a range in which an upper limit value is lower than an upper limit value of the fourth range and a lower limit value is lower than a lower limit value of the fourth range,
the sixth range is a range in which an upper limit value is lower than a lower limit value of the third range, and
the notification control unit provides, as notification, the second information related to a fourth state in which the worker is in the inactive state and the low-load state when the acquired activity level is within the fifth range and the acquired heart rate is within the sixth range.

9. The notification apparatus according to claim 8, wherein the storage unit stores (i) a seventh range of activity levels in a case where the worker is in an low-activity state where the worker is performing tasks involving a low activity level and (ii) an eighth range of heart rates when the worker is in an extremely low-load state, in which a load applied to the worker is lower than a load in the low-load state, in association with each other,
the seventh range is a range in which an upper limit value is higher than an upper limit value of the fourth range and lower than a lower limit value of the first range,
the eighth range is a range in which an upper limit value is lower than a lower limit value of the third range, and
the notification control unit provides, as notification, the second information related to the fifth state in which the worker is in the low-activity state and the extremely low-load state when the acquired activity level is within the seventh range and the acquired heart rate is within the eighth range.

10. The notification apparatus according to claim 1, wherein the activity level is represented by a ratio of energy consumed per unit time by the worker in the one state to energy consumed per unit time by the worker at rest.

11. The notification apparatus according to claim 2, wherein the notification control unit identifies that the worker is in a state corresponding to the one state during the work when a product of (i) the number of times the activity level acquired during a work period from a start time at which the worker begins the work to an end time at which the worker finishes the work is within the first range and the heart rate acquired during the work period is within the second range and (ii) the unit time reaches or exceeds the determination duration.

12. The notification apparatus according to claim 2, wherein the notification control unit sets the determination duration on the basis of a statistical value of a duration during which the activity level in each of a plurality of past periods was within the first range and the heart rate was within the second range.
